(19) United States
(12) Patent Application Publication
(10) Pub. No.: US 2004/0141152 A1
(43) Pub. Date: Jul. 22, 2004

(54) Apparatus and method for conducting vision screening

(76) Inventors: Joseph A. Marino, Park Ridge, IL (US); Steven Nordstrom, Hinsdale, IL (US)

Correspondence Address: FAY, SHARPE, FAGAN,, MINNICH & McKEE, LLP, 7th Floor, 1100 Superior Avenue, Cleveland, OH, 44114-2516, US

(21) Appl. No.: 10/674,110

(22) Filed: Sep. 29, 2003

Related U.S. Application Data
(60) Provisional application No. 60/414,161, filed on Sep. 27, 2002.

(51) Int. Cl. A61B 3/02

(52) U.S. Cl. 351/222

(57) ABSTRACT

A method and apparatus for conducting a vision-screening or test are provided. The method includes displaying a vision-screening or test on a display device that is initiated by a controller such as a handheld keypad controller, which is operatively associated with the computer-processing unit. The method also includes interactively completing the vision screening or test without the intervention of a health care professional.

This application relates to and claims the priority benefit of U.S. provisional patent application Serial No. 60/414,161 filed Sep. 27, 2002, and also claims priority to U.S. patent application Ser. No. 10/398,314 filed Apr. 4, 2003 based on International Application No. PCT/US02/08824 filed Mar. 21, 2002 which claims the priority benefit of 60/277,691 filed Mar. 21, 2001 the details of each are incorporated by reference.

### BACKGROUND OF THE INVENTION

The present invention relates to the screening or testing of vision. It finds particular application in conjunction with screening of a patient's eyes in a general health examination to identify the possibility of certain astigmatisms, eye diseases, and disorders. However, it is to be appreciated that the present invention is also amenable to other like applications, including, but not limited to ophthalmic examinations, pre-surgery evaluations, and ophthalmic research studies.

Numerous arenas exist where eye examinations or screenings take place, including but not limited to, general health examinations, periodic eye screenings or ophthalmic examinations, and pre-surgical eye examinations or eye evaluations within the scope of a research study (e.g. testing a new pharmaceutical drug or new medical procedure). In a vision screening, a general health care or school health professional must complete several screening tests in order to assess vision, including the possible presence of astigmatisms, amblyopia, and other eye diseases and disorders. Today, less than 25% of children entering kindergarten have had a vision screening. It is estimated that of these 25% of children who receive screenings, less than 5% are screened accurately. This raises a significant concern among healthcare professionals, since several conditions can become irreversible as the child becomes older. These vision conditions can present a threat to the quality of life and the child's ability to maximize school performance. Therefore, a vision-screening test is very important in detecting possible vision problems.

In those cases where a vision-screening test is conducted, typically a patient is positioned at a set distance from an eye chart, which is typically mounted on a wall. The patient will read letters or describe pictures seen on the eye chart. Visual acuity then measures the smallest line of text that the patient is able to read clearly. The test is usually conducted at the office of a health care professional who will ask the patient to sit or stand about twenty feet from the eye chart mounted or displayed on an opposing wall. The patient is instructed to remove any contact lenses, eyeglasses, and all other corrective vision apparatus. The health care professional covers one eye of the patient while the patient attempts to read the chart, and the patient continues to read down the chart to smaller and smaller letters until the patient is no longer able to read the letters because the letters appear too blurry. This process can be slow and difficult to gain the cooperation of the patient. Oftentimes, children get confused as to which line is being tested, which results in errors in the testing process. Less than 40% of referrals to pediatric ophthalmologists are determined to be accurate. Screenings can then take in excess of five minutes, particularly where a younger child becomes bored and does not cooperate. In instances where the child ceases to cooperate, it is not uncommon that the clinician can lose track of the progress or results of the test, once again creating the possibility for error. And, in a general health examination setting, the individual conducting the tests does so infrequently, making the likelihood of error or inconsistent testing protocols higher than in a setting where they are conducted on a daily basis. Inconsistencies in physical eye chart integrity or projected images of eye charts including discoloration, letter/symbol inconsistencies, lack of symbol validation, and room or projector illumination can individually or collectively adversely affect screening results. Additionally, all of these enumerated issues and more arise whenever a step by step eye testing protocol is used in conjunction with an image projected or printed on an eye chart, not just when visual acuity is measured.

Some health care professionals use automated vision screening devices or equipment that perform physical measurements of the patient's eye to identify risk factors associated with certain eye disorders. The use of automated vision screening equipment requires the patient to sit extremely still and avoid blinking of the eye. These devices, although expedient, have been identified as generating some false positives and false negatives as well as the fact that they are very expensive. Specifically, these devices cannot identify or measure the brain's interpretation and sensory interpretation of the character, letter, or symbol being viewed. Also, since the devices identify risk factors as opposed to the actual eye disorders and there is not a clearly documented correlation between the risk factors and the eye disorder itself, thereby, the false negatives and positives mentioned above may result.

It is known, for example, to provide a visual field test that requires direct patient interaction. Specifically, specialized test equipment uses a number of lights that individually flash or blink at different locations in the patient's field of vision. The patient is asked to record his/her perception of the light flash by, for example, depressing a handheld recorder. Thus, a binary response is the extent of the patient input to the test, i.e., the patient either perceived the light flash and recorded the event by depressing the recorder, or the patient did not perceive the light flash and the stimulus went undetected or unrecorded.

Although this specialized equipment runs one type of test and is automated in the sense that a sequence or pattern of light flashes are displayed to the patient and the patient provides a response thereto, the interaction with the patient is limited (binary response). Moreover, there is an inability to run a variety of tests on the specialized equipment. Further, the nature and extent of the information collected from the visual field test is limited. This equipment can conduct this procedure solely with the patient; however, the equipment is large, extremely costly, and dedicated to this single eye test. Accordingly, a need exists for patient interactive, automated, and/or vision screening or test methods and apparatus/systems for performing such vision screening or testing, particularly, a wide array of tests from a single system.

Therefore, it would be particularly desirable to provide a new method and apparatus for completing a vision screening or other vision tests that ensures an accurate and consistent approach to screening across health care, school health, researchers, and eye care professionals. The new method and approach should also be more economical than some of the automated equipment offered in the past and preferably be based on the accepted technology used within ophthalmic examinations, which would ensure a correlation of results between health care professionals. In addition, having the patient interact directly with the vision screening or testing equipment (without intervention of the health care professional, i.e., automating the test protocol and eliminating clinician discretion) would be preferable to reduce the prospects for errors and inconsistencies. Finally, this new method and apparatus should be able to offer a wide variety of visions screens or tests, so that a single piece of equipment could provide much of what a clinician would require in an examination.

### SUMMARY OF THE INVENTION

A method for conducting a vision screening or testing of the patient's eyesight is provided. The method includes displaying a vision screening test on a display device, such as a monitor. The vision screening test is initiated by a controller which is operatively associated with a central-processing unit (CPU). The method also includes the use of standard protocol or script for completion of the vision screening test, which runs automatically for the individual conducting the vision screening test. Furthermore, this method completes the vision screening test protocol interactively with the responses of the patient, which are entered on the controller.

In a preferred embodiment, a vision testing/screening (VT/S) apparatus according to the present invention includes a CPU with an associated display device. The VT/S apparatus further includes a controller such as a handheld keypad controller that is operatively associated with the CPU. The input devices in the preferred embodiment have been found to offer better functionality and ergonomics in the typical examination room. This hardware is combined with custom software, which accurately conducts the vision screening tests in accordance with a testing protocol established by the ophthalmology community, which also includes protocols developed for a research study of the eye as developed by a firm such as a pharmaceutical company or ophthalmic researcher. All of the procedural steps required to complete, for example, a vision screening, are pre-established and can be maintained within the CPU of the system. Thereby, upon start-up of the selected vision screening test, the clinician no longer needs to track or record the test results; the system completes the proper test protocol each time and records each and every response, and compares the response to the existing standards in accordance with ophthalmologic standards such as the American Academy of Pediatric Ophthalmology and Strabismus (AAPOS) and American Academy of Pediatrics (AAP) guidelines.

In accordance with commonly owned International Patent Application Serial No. PCT/US02/08824, filed Mar. 21, 2002, the details of which are expressly incorporated herein by reference, a system was designed to allow customization to a health care professional's testing environment including the length of the exam room, and the specific screening tests or screening protocols to be utilized. An associated process includes initiating a vision screening test from a CPU using a controller. The vision screening test is then displayed on a display device that is operatively associated with the CPU. Furthermore, a controller is used to respond interactively with the patient. The patient holds the controller, views what appears on the monitor, determines what optotypes he/she sees on the screen, and then presses the corresponding key on the controller. Alternatively, a clinician is able to input the patients' verbal or non-verbal responses. The system will assess whether the patient has correctly or incorrectly responded to what was actually on the monitor. Testing progresses in this manner and new optotypes appear on the monitor in accordance with the pre-programmed protocol. At the end of the screening, a pass/fail rating and near vision risk factor are presented for the patient based upon AAPOS and AAP guidelines, for example, or predetermined acuity levels by age group that are preferably incorporated into the protocol. Additionally, if a full visual acuity test or other type of eye test is selected, then an actual visual acuity level or other test result such as contrast or depth level is presented. This rating or acuity level is then utilized by the healthcare, school health professional, or other test administrator, to determine whether further action is appropriate or to simply record the results as a part of the data collection, i.e. a medical research study.

Still other advantages of the present invention will be recognized upon a reading and understanding of this specification by one of ordinary skill in the art. For example, any assessment, screening, test or evaluation of the eye whereby a patient is asked to respond to what is presented on a chart or screen, will benefit from this controlled, automated testing approach and apparatus.

### DESCRIPTION OF THE DRAWINGS

The present invention may take form in various components and arrangements of components, and/or in various steps and arrangements of steps. The drawings are only for purposes of illustrating preferred embodiments and are not to be construed as limiting the invention.

FIG. 1 illustrates a preferred embodiment of the vision screening or testing system, including a CPU, a display device, a controller, a printer and software.

FIG. 2 illustrates one embodiment for a keypad controller for control of the vision screening or testing system.

FIG. 3 is a flow diagram illustrating a routine for establishing a vision testing or screening protocol.

FIG. 4 is a flow diagram illustrating a routine for listing the vision screening tests and charts available on the system or to exit the system.

FIG. 5 is a flow diagram illustrating a routine to run a vision screening or testing protocol and capture all patient responses for permanent record on diskette, hard drive or printed.

FIG. 6 is a flow diagram illustrating a routine for manually utilizing a chart of randomly generated optotypes and manually adjusting their acuity level using keys.

FIGS. 6A-6C show a single line of letters and an optotype pointer that may be used as part of a vision screening test.

FIG. 7 is a flow diagram illustrating a routine for exiting.

FIG. 8 is a flow diagram illustrating a routine for reconfiguring the system for changes in the testing environment, including but not limited to, calibration distance, change in input device in conjunction with a change in number and types of vision tests needed.

### DETAILED DESCRIPTION OF THE PREFERRED EMBODIMENTS

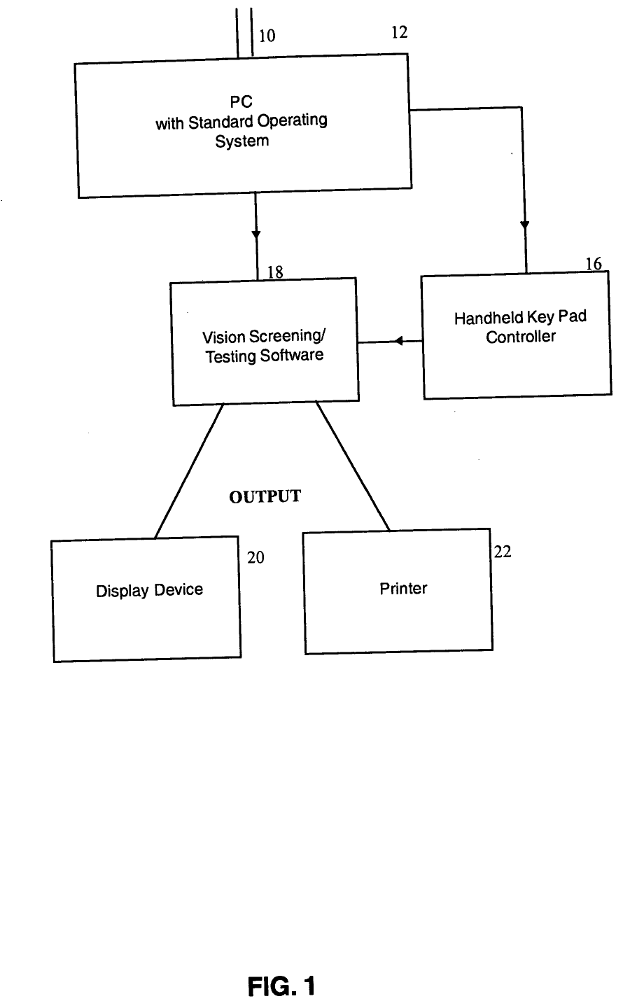

In accordance with one aspect of the present invention, an apparatus and method for conducting a vision-screening test is provided. FIG. 1 schematically illustrates a computer-based apparatus or system in accordance with a preferred embodiment of the present invention. The system includes a conventional computer or central processing unit (CPU) 12 with an operating system, such as Windows®. It is contemplated, however, that the present invention can be designed to be operate within other computer operating systems, such as Apple®, Linux®, UNIX and so forth. The system includes a display device 20 used in conjunction with the computer 12, a controller 16 to input information, such as the illustrated handheld keypad, used in conjunction with control software 18 to provide video display, for example, via serial cabling 10. It is understood that other input devices can be utilized in this invention, including but not limited to, a conventional computer keyboard, conventional computer mouse, infrared remote control device, RF remote control device, joystick, handheld computer, etc. The method according to the present invention includes the use of a particular arrangement of components for conducting a vision-screening test. Preferably, customizable data, images, and audio are displayed to a patient using a conventional personal computer including the display monitor 20 and a printer 22 operatively associated therewith in order to conduct a vision screening. It is contemplated, however, that the present invention may be relevant to practice in other applications throughout the healthcare field, and as such, the following description of the present invention should not be construed as limiting the present invention in any way.

The software 18 allows each health care professional to individually configure the vision screening tests. This is accomplished by establishing a process file that contains parameters that define both the behavior and the appearance of the system. Preferably, during initial set-up of the vision screening system, the health care professional specifies the vision screening test and other eye charts required on the system. These assignments are then entered into process files during set-up of the system. At the same time, the actual vision screening or testing protocols are loaded into the system so that the screening or tests are conducted automatically and interactively with the patient's responses. As will be appreciated, the terms screening or testing are used generally synonymously, and reference to one term should apply to the other without limiting the scope and intent of the present invention.

In addition, the health care professional may reconfigure the system into a fall-scale eye testing system with the complete customization features available in that system, as fully disclosed in PCT/US02/08824.

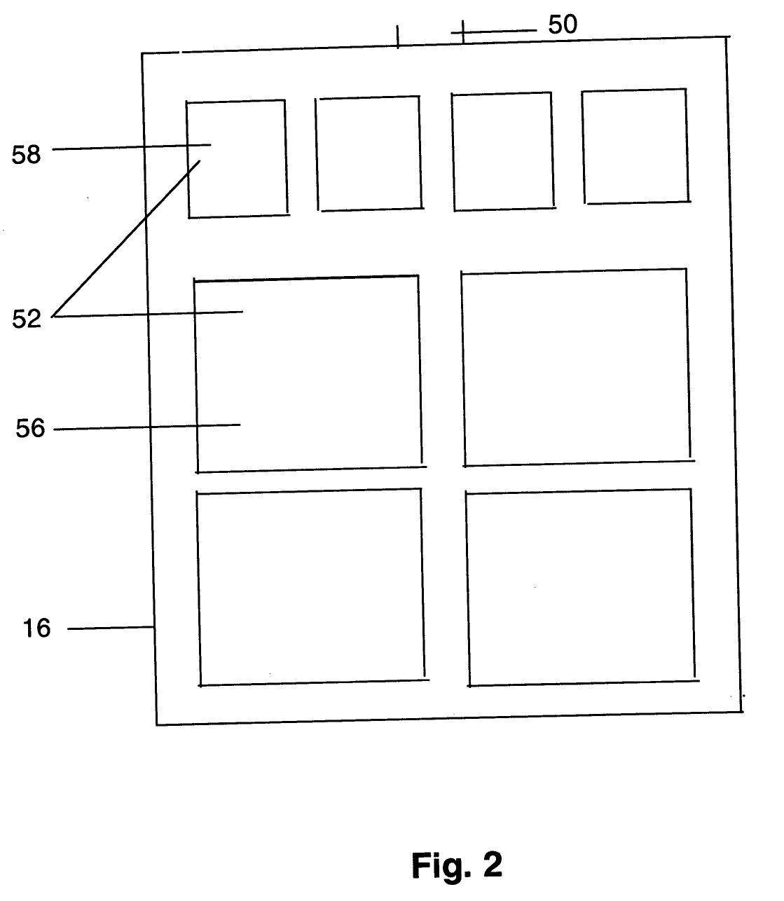

FIG. 2 illustrates one embodiment for a handheld keypad controller 16 in accordance with the present invention. The keypad controller is the device that initiates the vision screening tests that are administered. As the origination point of clinical screening, the keypad controller interprets an individual scan code when a key from the keypad 52 is depressed and then sends the required information to the display device associated with the personal computer. Throughout the screening process, as keys are depressed by the patient, these individual scan codes will be interpreted and the screening protocol will be sequentially executed. As shown, the keypad unit 52 is an electromechanical device. A connection is made to a serial digital I/O port 50. The keypad controller is located in the examination room within the immediate proximity of the health care professional and the patient.

The keypad controller is connected to a digital I/O port. The keypad controller has, for example, a performance capability of accessing an unlimited number of independent screening tests, single line acuity tests, or other vision tests utilizing contact square surface area keys 56. The keypad is preferably handheld, however, it is contemplated to alternatively be used as a tabletop controller or may adapt still other configurations without departing from the scope and intent of the invention. The keypad is designed in the exemplary embodiment so that the system operational keys 58 are separated from the interactive keys 56 used in the vision screening. Additionally, the vision screening or interactive testing keys are larger (approximately four times that of a normal key) for ease of use during interactive screening.

As shown, the handheld keypad controller has preferably about eight individual keys, although it is contemplated that any number of keys or tests can be included in the keypad. The keys within the keypad 52 are used to display at least the following functions: four operating keys 58 1) identify vision screening tests and eye charts 2) select vision screening tests and eye charts 3&4) scroll up or down through available screening tests and through acuity levels on an eye chart. Four interactive keys 56 are used by the patient to depress the key, which reflects the optotype, the patient sees on the monitor during the course of a vision-screening test. These interactive keys can either be directly labeled with the response option available to the patient or a template can be overlaid upon the keys upon the start of testing. Additionally, the protocol itself can redefine keys at different points during the running of a screening or test, if needed. Of course it should be appreciated that the keypad controller is preferred, although alternative controllers that achieve the same functions and advantages offered by the present arrangement can be used without departing from the scope and intent of the present invention. For example, it is contemplated that a personal digital assistant (“PDA”) can be adapted for use in the present invention as a keypad controller to be held by the health care professional in initiating the visual acuity screening program.

The vision testing/screening program (“VT/SP”) is a computer program that runs scripted vision testing/screening protocols through a script interpreter and displays standard eye charts or subsets thereof including, pictures, symbols, letters, tumbling “E's, sinusoidal gratings, low contrast letters/symbols, color charts etc. The invention has been developed so that any test of the eye that is ordinarily displayed on a chart, monitor or other display device could be run on the VT/SP, including but not limited to visual acuity, vision screening, depth perception, low contrast, contrast sensitivity, color blindness, duo chrome, etc. The calibration of the components is based on the distance from the display device to the patient. The input of this measurement is required only once in the initial setup, and the system can be recalibrated as explained below.

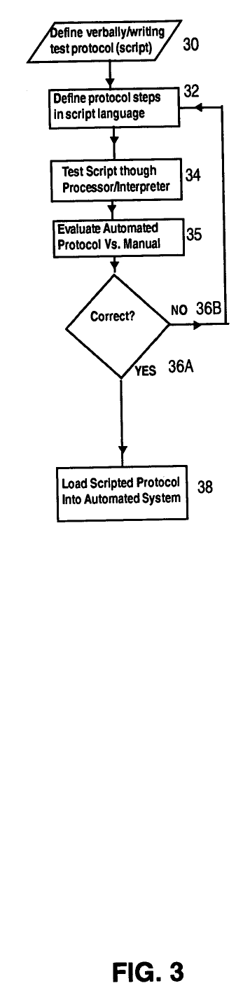

FIG. 3 illustrates one embodiment of a routine that utilizes a script language and interpreter to develop an automated eye testing protocol that can run on any computerized or electromechanical display device. The establishment of an automated eye testing protocol is initiated by an eye care professional or other interested party (such as a researcher) defining each step taken for the specific eye test or screening 30. The eye care professional defines each of the eye test or screening steps, including but not limited to, switching optotypes and acuity levels or contrast levels. Also, the various evaluations and notations of results that occur throughout the testing process are defined. Once documented, the test protocol (script) is developed in any computer scripting language that has operational steps to enable the execution of the protocol correctly as represented at step 32. The computerized script of the eye test protocol is then executed as represented at step 34 and evaluated with the script processor that would be used in day to day use of the protocol on the automated apparatus presented in FIGS. 1 and 2. Automated results are compared to the results that would have been obtained from a precise manual execution of the test protocol as represented at step 35. If the automated results match the results that would have been returned from a precise manual execution of the test protocol 36A, no further revisions are required and the computerized script is loaded onto the apparatus in FIG. 1 for use in day to day examinations 38. If the results do not match, then the computerized script is revised and retested until it is correct 36B.

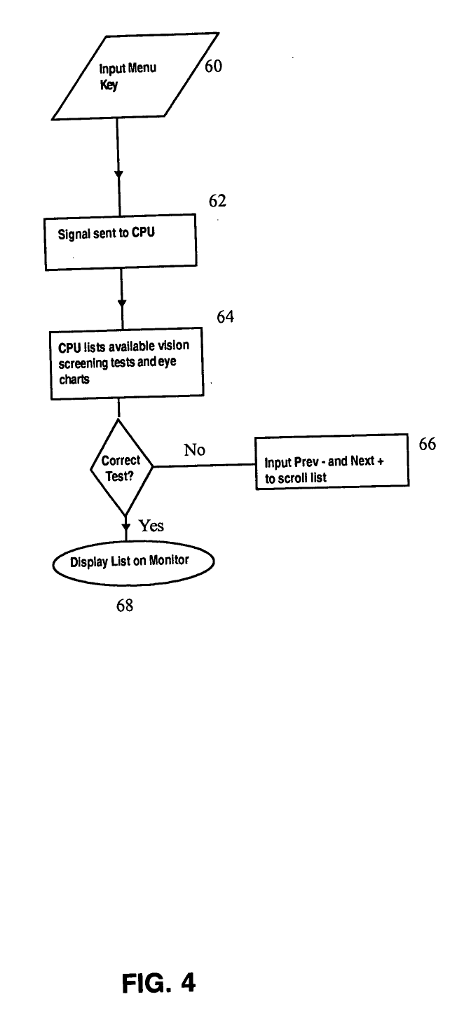

FIG. 4 illustrates one embodiment of a routine that identifies and lists all vision screening tests and eye charts available on the system. The screening test or chart is identified by the health care professional by depressing the “Menu” key 60 on the custom keypad controller 16. This action causes the software 18 to access all vision screening tests available 62 as generally represented by reference numeral 64 on the system. Then, the health care professional can selectively scroll as represented at flow chart step 66 to the desired acuity screening test by depressing either the “Menu” or “Age” keys 58 (FIG. 2). The description of the available screening tests and eye charts then appears in large print or menu format at the bottom of the display monitor 20 as represented by 68.

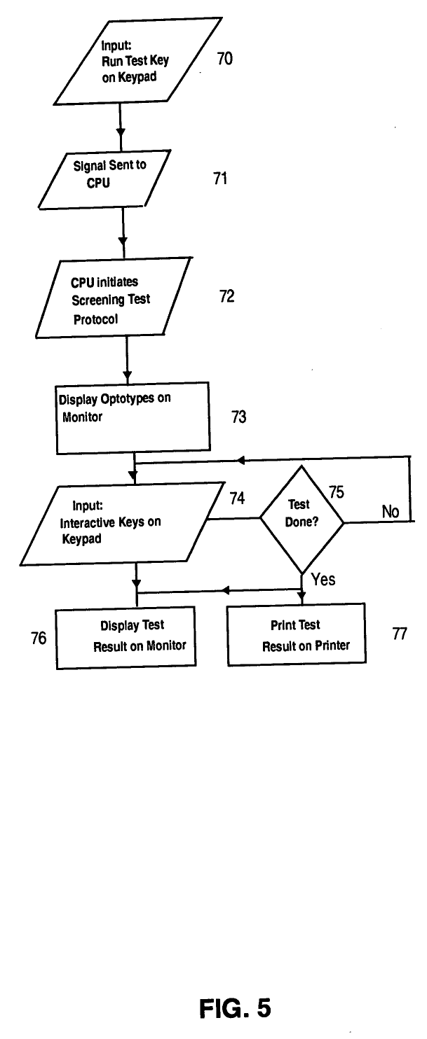

FIG. 5 illustrates one embodiment of a routine that initiates and operates an automatic vision screening test protocol. As previously discussed, the test may be of other aspects of vision including, but not limited to, low contrast, depth perception, glaucoma, or still other tests. The automatic vision screening test protocol is initiated by the health care professional depressing the “Run Test” key 70 on the controller or keypad 16 following selection of the desired vision-screening test. This action 71 causes the software to conduct an interactive screening test with the patient 72. The health care professional will hand the keypad to the patient who is requested to depress the key that coincides with the optotype that appears on the monitor 73. Following each keypad input 74, the software evaluates at step 75 the patient's response and determines whether it is correct or incorrect. It is also contemplated that the time of response may be evaluated, in addition to the software determining whether the response is correct/incorrect. Likewise, if the patient takes too long to respond, the software may record this as an incorrect response. Then the software will proceed through the protocol altering the acuity levels and adding additional optotypes in accordance with the defined protocol for the screening test. At the completion of the vision screening protocol, the software tabulates the results and either presents on the monitor 76 and/or printer 77, a visual acuity level for each eye or a pass/fail at a designated acuity level. Which end result is presented is dependent on the vision screening test selected and what that protocol requires. It is also contemplated that the results can be stored for later access or forwarding, for example, in an electronic format to a remote location if desired.

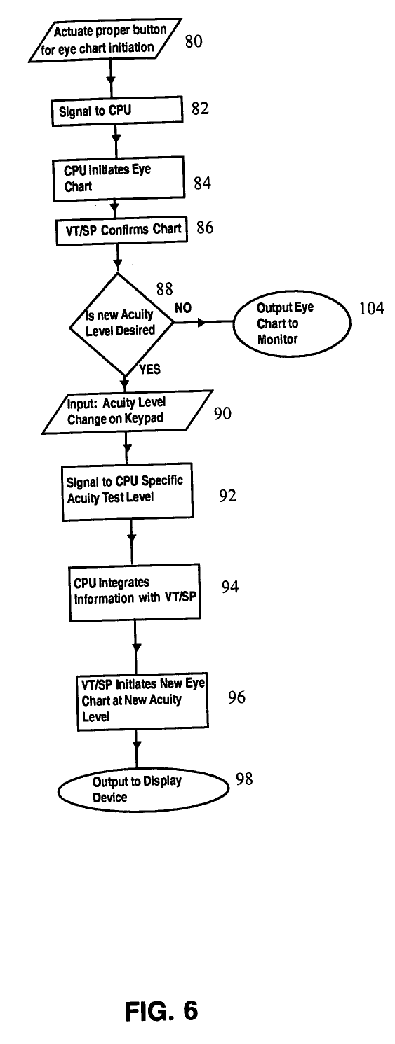

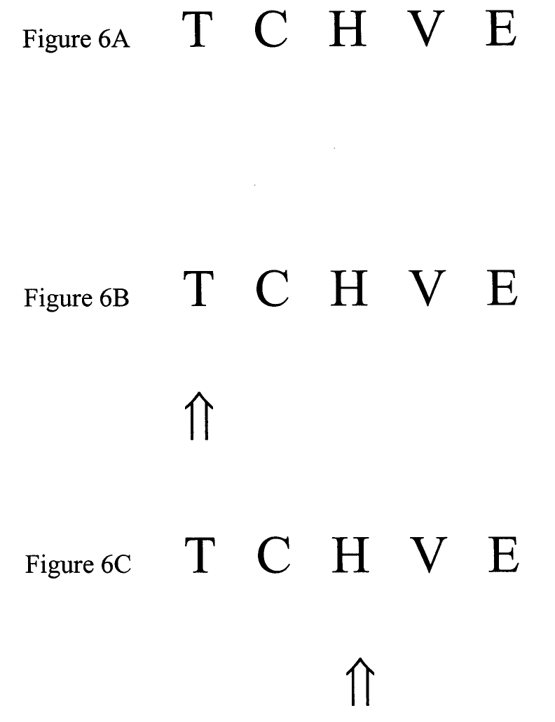

FIG. 6 illustrates one embodiment of a routine that initiates a visual acuity chart in accordance with the present invention. The visual acuity chart is initiated by the health care professional depressing one of the healthcare professional's specified acuity type test keys on the custom keypad controller in step 80. Each key is preferably dedicated to one or more tests, for example one or more of the following acuity tests: letters, pictures, tumbling E's and HOTV (all are customary acuity tests, although the list should not be restrictive since newly developed tests may also be accommodated). Upon actuation (e.g., pressing) of a key, the signal is sent to the CPU in step 82 and the software translates the signal from the keypad into the desired visual acuity chart in step 84, 86. The health care professional decides whether another acuity test level is required. If no new acuity level is desired, the VT/SP will instruct as represented at step 88 the test level to be presented on the display device as referenced at 104. The software is normally programmed to remember the last acuity test level that was presented and can incorporate both the test and level into its instructions to the CPU. The desired visual acuity test is displayed on the display device as per the design of the health care professional at the acuity level last presented in an acuity test. If a new acuity test level is desired, the health care professional can select any available acuity test levels by selecting the keys to move down or up, respectively, in acuity level as noted in step 90. The signal to change the acuity level is relayed to the CPU in step 92, which integrates that information with the software in step 94 to initiate a new visual acuity chart with a new acuity level in step 96. Each eye chart at the selected acuity level is then displayed on the monitor 98. Each optotype will appear in a line and an optotype pointer (FIGS. 6A, 6B and 6C) will appear under the furthest left character in the line.

It can be advanced on the line using predetermined keys such as the Menu and Age keys, which simultaneously tracks whether the patient's response was correct or incorrect. The use of an optotype pointer to assist in conducting examinations is essentially the same as that disclosed in PCT/US002/08824.

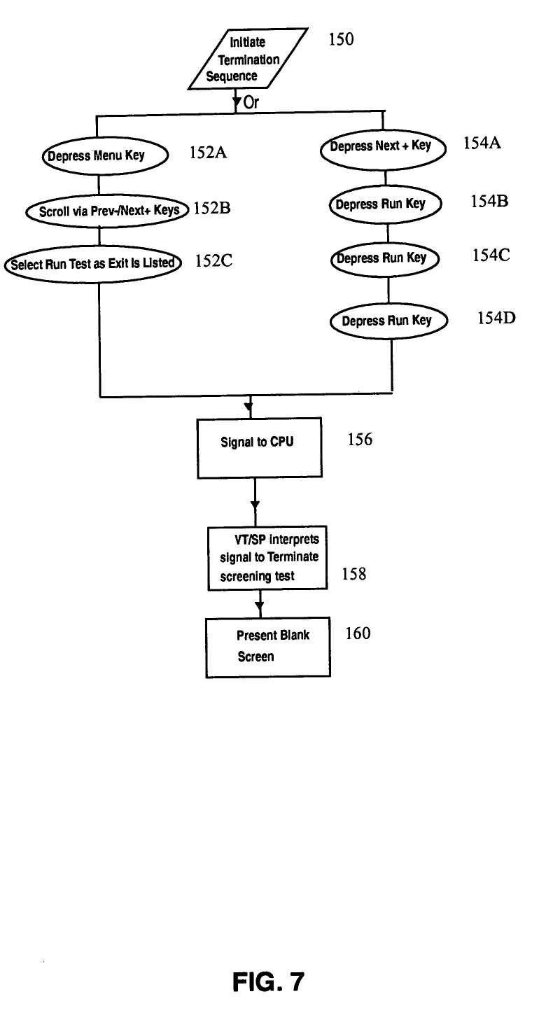

FIG. 7 illustrates two examples of a typical exit routine for the vision screening system in accordance with the present invention. The shutdown of the system is initiated at 150 by the health care professional depressing a pre-defined sequence of keys on the custom keypad controller. One example is that the healthcare professional can depress the “Menu” key 152A followed by scrolling to “Exit” 152B using either the Menu or Age key then selecting “Exit” with the “Run Test” key 152C. The system can also be shut down when the CPU is simply turned off manually. Additionally, the vision screening system can exit a screening protocol midway through it by simply depressing the Menu 154A, “Run Test” 154B, “Run Test” 154C, “Run Test” 154D keys in sequence or a user-designated sequence of keys. The signal 156 is sent from the controller and interpreted by the vision testing/screening software 158. The vision testing/screening software then signals the display device at step 160 to present a blank screen. This is particularly useful when a physician or medical assistant selects the wrong screening test and needs to move into another one without taking the time to finish the actual screening protocol.

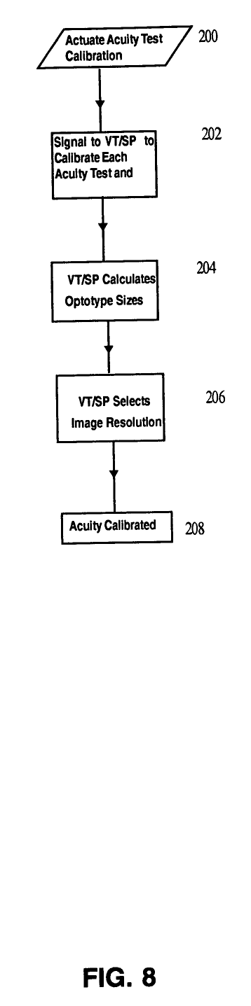

FIG. 8 shows a preferred embodiment of how each of the acuity tests is calibrated to the size of the rooms. The calibration action is typically initiated prior to receipt of the system by the healthcare professional but can also be initiated by the health care professional. Calibration is typically done by utilizing a conventional keyboard to input as represented in step 200. Using the keys, the examination room size is input into CPU memory. Once input, the software is signaled to calibrate each vision screening test and eye chart in step 202. The software will take this input and begin calculations on each of the vision screening tests and charts at each acuity level to ensure that each test and measurement precisely displays the size of the test, picture or specific optotype in accordance with the desired acuity level at the time of the examination in step 204. The software can store the calibration information and utilize this information every time it instructs the CPU which vision screening test or eye chart to initialize.

Based on the room length entered above, the software precisely calculates in twips (a standard unit of graphic size that is independent of resolution on a monitor) the height and width of the characters. During the initial set up process, one of several resolution options is preferably automatically selected through the software, ensuring that the appearance of each letter or image is the best for each size 206. Several image files of varying resolution level are included with the software and are stored within the hard drive of the CPU. Using the length of the room and the size of the optotype in twips, the image file, which is stored on the hard drive, can then be increased in size or decreased in size with the clearest image being automatically selected by the software. The software will then perform a validation of the image selection by proofing the mathematics of the required image adjustment from the selected image file. If an alternate image file, however, interpolates better mathematically, the selected image file will be revised. The software uses interpolation and calculation for sizing the images, which is used in combination with the automatic resolution selection at set up. The software permanently stores the calibration and utilizes this information every time a vision screening test or eye chart is initiated 208. This capability is as was described in PCT/US02/08824.

In addition to being able to calibrate a vision screening test or eye chart depending on the size of the room, different parameters can be set during the initial set up of the system. Different vision screening tests, vision screening protocols, the selected letters or pictures used with each screening test or chart, and the line configurations including randomization are examples of the types of parameters that can be set up initially by the health care professional. The parameters are entered into the software during the initial set up using a conventional keyboard. The parameters are then interpreted by the software and stored on the hard drive of the CPU. Following the parameter selection by the health care professional, the software interprets the critical size parameters (e.g. room length for testing) and selects an optimal image resolution size for each letter, picture, and optotype that is stored as a preference and is used each time the particular vision screening test and eye chart to which the parameter is correlated is initiated. This capability is further described in PCT/US02/08824.

The invention has been described with reference to the preferred embodiments. Obviously, modifications and alterations will occur to others upon a reading and understanding of this specification. It is intended that the invention be construed as including all such modifications and alterations insofar as they come within the scope of the appended claims or the equivalents thereof.

What is claimed is:

1. A method of testing a patient's vision comprising the steps of:
displaying a vision test on a display device, wherein the vision test is stored on a central processing unit and the display device is operatively associated with the central processing unit;
having a patient input a response from at least two choices into a controller in response to the display on the display device, wherein the controller is operatively associated with the central processing unit; and
storing patient actuated responses to the vision test.

2. The method of claim 1, further comprising the step of running a vision test protocol stored on the central processing unit.

3. The method of claim 2, further comprising the step of changing the display of the vision test in response to the patient input according to the vision test protocol.

4. The method of claim 3, further comprising the step of repeating the steps of having a patient input a response, and changing the display of the vision test according to the vision test protocol.

5. The method of claim 4, wherein the vision test protocol in the running step includes each step in a visual testing program in accordance with standards of the ophthalmologic community.

6. The method of claim 1, further comprising the step of determining whether the patient actuated response is correct.

7. The method of claim 6, wherein the step of determining whether the response is correct is performed automatically by the vision test protocol.

8. The method of claim 1, further comprising the step of automatically changing the display of the vision test in response to the patient input.

9. The method of claim 8, wherein the step of automatically changing the display further comprises changing the location of a pointer in relation to an optotype.

10. The method of claim 8, wherein the step of automatically changing the display further comprises changing the contrast of the display.

11. The method of claim 1, comprising the further step of displaying vision test results.

12. The method of claim 11, wherein the step of displaying vision test results further comprises the step of displaying a pass/fail rating for the patient based upon predetermined acuity levels.

13. The method of claim 1, wherein the step of displaying vision test results further comprises the step of displaying a visual acuity level.

14. The method of claim 1, wherein the step of displaying vision test results further comprises the step of displaying one of a visual contrast and depth perception level.

15. A method of testing a patient's vision comprising the steps of:
displaying on a display device one of plural vision tests stored on central processing unit operatively associated with the display device;
running a vision test protocol corresponding to the vision test displayed on the display device;
receiving a response into a controller in response to the display displayed on the display device, wherein the controller is operatively associated with central processing unit; and
changing the display of the vision test according to the vision test protocol.

16. The method of claim 15, further comprising the steps of
developing the vision test protocol for the vision test; and
loading the vision test protocol onto the central processing unit.

17. The method of claim 16, wherein the step of developing the vision test protocol step further comprises the step of developing the vision test protocol in accordance with standards of the ophthalmologic community.

18. The method of claim 15, further comprising repeating the step of having a patient input a response and the step of changing the display of the vision test in response to the patient input according to the vision screening test protocol.

19. The method of claim 15, wherein the step of changing the display further comprises the step of changing the location of a pointer in relation to an optotype.

20. The method of claim 15, wherein the step of changing the display further comprises the step of changing the contrast of the display.

21. The method of claim 15, wherein the step of receiving a response further comprises having a patient input a response into the controller.

22. The method of claim 15, wherein the step of receiving a response further comprises having a clinician input a response into the controller in response to one of verbal and non-verbal indications by a patient.

23. An apparatus for testing a patient's vision comprising:
a display device for displaying a vision test;
a central processing unit operatively communicating with said display device, said central processing unit including the vision test and a corresponding protocol for the vision test; and
a controller operatively communicating with said display device.

24. The system of claim 24, wherein the corresponding protocol includes each step of the vision test.

25. The system of claim 24, further comprising means for automatically changing the vision test displayed on said display device according to a response received from said controller.

26. The system of claim 24, wherein the vision test includes a display of optotypes on said display device.

27. The system of claim 26, wherein the protocol changes the display of optotypes in response to input received by said controller.

28. The system of claim 24, further comprising means for storing input received from said controller.

29. The system of claim 28, wherein the protocol determines a patient's pass/fail rating from input stored in said storing means using standards developed by the ophthalmologic community.

30. The system of claim 28, wherein the protocol determines a patient's visual acuity from input stored in said storing means using standards developed by the ophthalmologic community.

31. The system of claim 28, wherein the protocol determines at least one of a patient's low contrast and depth perception from input stored in said storing means using standards developed by the ophthalmologic community.

32. A vision test protocol for use with a vision test apparatus wherein the apparatus includes a display device, a central processing unit operatively communicating with the display device, and a controller operatively communicating with the display device, wherein the central processing unit stores more than one type of vision test, the vision test protocol comprising means for automatically changing at least a portion of a vision test displayed on the display device in response to input received by the controller.

33. The apparatus of claim 33, further comprising means for recording input received by the controller.

34. The apparatus of claim 33, further comprising means for providing test results from the input stored in said input recording means.

35. The apparatus of claim 34, further comprising a program that contains each step of a vision test in accordance with standards developed by the ophthalmologic community.
